(19) United States
(12) Patent Application Publication
(10) Pub. No.: US 2024/0398367 A1
(43) Pub. Date: Dec. 5, 2024

(54) SUPPORT FRAME FOR AN ANGIOGRAPHY SYSTEM

(71) Applicant: Siemens Healthineers AG, Forchheim (DE)

(72) Inventors: Marius SEITZ, Pressath (DE); Ralf GAERTNER, Kemnath (DE); Stefan SCHMIDT, Stoernstein (DE)

(73) Assignee: Siemens Healthineers AG, Forchheim (DE)

(21) Appl. No.: 18/675,549

(22) Filed: May 28, 2024

(30) Foreign Application Priority Data
May 30, 2023 (DE) 10 2023 205 041.2

(51) Int. Cl. A61B 6/50; A61B 6/00

(52) U.S. Cl. CPC A61B 6/504; A61B 6/4441; A61B 2560/0406

(57) ABSTRACT

A curved support frame for an angiography system comprises a hollow body having a first frame element and a second frame element, wherein the first frame element and the second frame element together form a closed lateral area of the curved support frame. An angiography system including the curved curved support frame is also provided.

### CROSS-REFERENCE TO RELATED APPLICATION(S)

The present application claims priority under 35 U.S.C. § 119 to German Patent Application No. 10 2023 205 041.2, filed May 30, 2023, the entire contents of which is incorporated herein by reference.

### FIELD

One or more embodiments of the present invention relate to a support frame for an angiography system and also to an angiography system.

### BACKGROUND

Angiography systems are medical examination or treatment facilities and have a C-arm on which examination or treatment devices, for example an x-ray source, a diaphragm and also an x-ray detector are arranged, which are able to be moved in the room via the C-arm. X-ray source and x-ray detector serve to record fluoroscopy images, before, after or during a medical intervention on a patient for example. The C-arm is usually arranged on a movement facility, for example a stand mounted on the floor or suspended from the ceiling or a multi-axle articulated arm. A support arm and/or an intermediate carriage can further be provided between stand and C-arm, so that the C-arm can be adjusted movably by a number of degrees of freedom in relation to its environment in the room in order to be able to position the equipment arranged on the C-arm with respect to the patient for a desired x-ray imaging process.

C-arm, support arm and/or intermediate carriage in this case typically have a curved shape, their shape is thus emulating a circumferential segment of a fictional circle. Expressed in different terms, their longitudinal axis runs on an orbital path or essentially on an orbital path.

The manufacture of a support profile for a C-arm, a support arm and/or an intermediate carriage is known, in that for example a small aluminum pressing is extruded and subsequently rolled into the desired orbital shape. As an alternative the profile is manufactured as a cast metal part, usually likewise made of aluminum or an aluminum alloy. Depending on the process, areas with increased and/or uneven wall thickness occur during casting, which have more material then would be necessary for the loads involved. An expensive machining is then required in order to achieve the desired dimensional accuracy.

As an alternative to this the manufacture of a support profile via welded modules is known, in which a type of box shape consisting of a number of sheets of metal is assembled. This process is very expensive, since due to the curvature of the profile many individual parts have to be welded. These have to be clamped into their defined relative position manually before the actual welding, which is very time-consuming and thereby personnel-intensive. Moreover, with a high number of parts, many weld points or weld seams and also where necessary their post processing are required, which likewise disadvantageously increases the assembly time.

### SUMMARY

In different examination or treatment methods the C-arm must be moved around the patient at high speed in order to record 3D images. Increasingly higher demands are made here as regards the rigidity and the increase in the eigenfrequency of the angiography system, in order to obtain the desired image quality and in particular smooth operation. In order to increase the rigidity of the C-arm the wall thickness could be increased, which on the one hand however renders the manufacture of the C-arm increasingly more difficult and necessarily also results in a significant increase in the weight of the system, whereby the moved mass would also increase. This would be at the expense of the wear behavior and the setup costs for drives and the like. Moreover the eigenfrequency of the system would be reduced, which adversely affects the stability of the C-arm movement.

By contrast with this, an object is to provide a support frame for an angiography system optimized in respect of its mechanical stability. A further object is to provide a support frame optimized in respect of its manufacturing cost.

At least this object is achieved by a support frame and also by an angiography system according to one or more embodiments of the present invention. Preferred and/or alternate, advantageous embodiment variants are also disclosed herein.

An embodiment of the present invention is directed in a first aspect to a curved support frame for an angiography system. This is embodied as a hollow body and comprises a first and a second frame element, wherein the first and the second frame element together form a closed lateral area of the support frame.

Advantageously the support frame simply comprises a supporting envelope or an envelope profile, i.e. the support frame is empty or hollow in its interior, so that the interior space is especially suitable for accommodating power supply and/or data transfer lines of the angiography system within it.

The supporting construction of the support frame is further formed or composed of just two parts, namely the first and the second frame element. This significantly reduces the number of individual parts during the production as well as the overall material requirement. The small number of parts also reduces the number of required joining steps, such as for example welding, as well as post or intermediate processing steps, such as for example folds.

In a further aspect, an embodiment of the present invention is directed to an angiography system comprising an inventive support frame. The angiography system comprises a C-arm, at the end of which an x-ray source or opposite it an x-ray detector are arranged, which act together for x-ray imaging. The angiography system can involve a floor-mounted or ceiling-suspended system or a system allowing mobile movement on the base. In versions the angiography system can be attached via a stand to the floor or to a ceiling of the room. Starting from this stand, at least one support arm can be provided, on which the C-arm is mounted. As an alternative the C-arm can be connected via a multi-axle articulated arm, preferably a robot arm, to a stand. In versions of the angiography system comprising a support arm, this is typically embodied as a curved connection unit between stand and C-arm, which is arranged for rotation at least one, preferably at two connecting points. In particular in one version comprising a support arm an intermediate carriage can additionally be provided, wherein this is then arranged between support arm and C-arm. In particular the intermediate carriage provides a degree of freedom of movement in which the C-arm moves along a curved longitudinal axis on an orbital path and accordingly can be rotated on the orbital path about the isocenter of the angiography system. Overall articulated arm, support arm and/or intermediate carriage in the various embodiments of the angiography system provide various degrees of translation and/or rotation freedom for the C-arm.

In a preferred embodiment of the present invention the C-arm of the angiography system comprises the inventive curved support frame.

In a further embodiment of the present invention the support arm of the angiography system comprises the inventive curved support frame.

In the same way the intermediate carriage can also be embodied with the inventive curved support frame.

Especially preferably both the support arm and also the C-arm as well as the intermediate carriage, if present, can be embodied via an inventive support frame.

The curvature of the support frame extends in a preferred embodiment along the likewise curved longitudinal axis of the support frame. The curved longitudinal axis preferably runs in this case on an orbital path. The curvature of the support frame accordingly preferably emulates a circular path or approximates to this, i.e. in embodiments the curvature of the support frame is always the same over its length. In other embodiments the curvature of the support frame can be different in sections of the circumference, i.e. there are at least two sections of the circumference, the curvature of which differs from each other. In these embodiments the support frame, in the mounted state, preferably forms typical orbital shapes for a C-arm, a support arm and/or an intermediate carriage.

The lateral area of the support frame formed by the two frame elements runs around its longitudinal axis. To put it differently, the curved longitudinal axis of the support frame runs in the center of the same and essentially passes at right angles through the bottom and top surface of the support frame not closed in a typical embodiment.

The lateral area formed by the first and the second frame element is closed, i.e. when the first and the second frame element assume their final relative position in relation to one another, the lateral area has no gaps, openings, holes or splits. The interior of the support frame is therefore, in a preferred embodiment, only accessible via the open bottom or top surface of the support frame.

The frame elements, in a preferred embodiment of the present invention, have an extent in which they extend over the entire length of the support frame, i.e. from its bottom surface through to its top surface. Their extent in the longitudinal direction is thus far greater than their extent running transverse thereto.

In a preferred embodiment the first and the second frame element each have a shape that, in the assembled state of the frame elements, in cross section forms a square or a rectangle. In other embodiments the cross-sectional shape can also deviate from said shape. For example the cross section of the lateral area can be embodied oval, elliptical or round or also in a mixed form. A rectangular shape however proves especially favorable compared to the other variants as regards the manufacturing method.

The first and the second frame element are preferably embodied in embodiments as follows: The first frame element comprises a first part surface and the second frame element for its part comprises a second part surface, wherein the first and the second part surface embody parallel side surfaces of the support frame lying opposite one another. In particular the first and the second part surface are embodied in such a way that they each extend over the entire length (along the curved longitudinal axis) of the support frame or of the frame elements. In other words the first and the second part surface form closed, i.e. contiguous surfaces of the support frame.

A side surface of the support frame refers in this case to an area of its lateral area that lies parallel to the radius of the fictional circle on the orbital path of which the longitudinal axis of the support frame runs.

Especially preferably the first and the second part surface are formed when they each comprise at least two trapezoidal surface segments respectively. In other words at least two, preferably more than two, trapezoidal surface segments are comprised by the first and also the second part surface in each case. In this case the respective at least two trapezoidal surface segments lie on one of the part surfaces with at least one of their respective limbs adjacent to one another. In other words two neighboring trapezes of the first and/or the second part surface comprise a common limb. The at least two trapezoidal surface segments are connected to one another via the common limb in one plane or are embodied in one piece with one another. The sides of the trapezoidal surface segments running in parallel together form outer edges of the first and the second part surface. The embodiment of the individual surface segments as trapezes allows these to be arranged at any given angle to one another, whereby one, or in some embodiments, a number of random curvatures, can be set for the support frame.

Especially preferably neighboring trapezoidal surface segments of the first and/or the second part surface are arranged in relation to one another at an angle in a range of 155° to 175°, preferably, in a range of 160° to 170°, especially preferably at an angle of 165° or 167°. In this way the desired curvature radii and dimensions of a C-arm, of a support arm and/or an intermediate carriage can be realized especially well.

The first frame element further preferably comprises in some embodiments at least one third and one fourth part surface, which each extend at right angles from the first part surface, in particular in order to embody a square or rectangular cross section of the support frame. The second frame element is then embodied in a similar way. It comprises at least one fifth and sixth part surface, which each extend at right angles from the second part surface. In especially preferred embodiments the first and the second frame element, as well as the first and second part surface, comprise more than two, i.e. at least three or four, further part surfaces. First, third and fourth part surface of the first frame element are embodied in this version in one piece or contiguously, as are the second, fifth and sixth part surface of the second frame element.

In this case it should still be pointed out that the third and fourth part surface as well as the fifth and sixth part surface do not touch each other directly, but are merely linked up via the first or the second part surface. To this extent, when the first and the second frame element are arranged in their final position relative to one another, part surfaces of the first frame element come into contact with part surfaces of the second frame element in such a way that for example a part surface of the first frame element is surrounded by part surfaces of the second frame element or a part surface of the second frame element is surrounded by part surfaces of the first frame element.

The at least third, fourth, fifth and sixth part surface in this way each form the remaining sides of the closed rectangular lateral area of the support frame.

In this case the third and fourth part surface of the first frame element are connected in each case to one of the trapezoidal surface segments of the first part surface. In other words the third and fourth part surface of the first frame element each adjoin one of the respective parallel sides of a trapeze of the first part surface. The fifth and sixth part surface of the second frame element are each connected to one of the trapezoidal surface segments of the second part surface. In other words the fifth and sixth part surface of the second frame element each adjoin one of the respective parallel sides of a trapeze.

As a result, third, fourth, fifth and sixth part surface each do not extend over the entire length of the support frame (from a top to a bottom surface), but have a length essentially parallel to a longitudinal axis tangent, which corresponds to the length of the parallel side of the assigned trapezoidal surface segment.

In an especially preferred embodiment of the present invention, the first and the second frame element are each embodied with their various part surfaces as bent metal sheets. This embodiment has the advantage that the frame elements are first cut by laser cutting or by a punching method from a flat metal sheet and subsequently the at least third, fourth, fifth, sixth part surface are bent about the parallel sides of the trapezoidal surface segments of the first and second part surface. Metal sheets can be obtained with small dimensional tolerances in various thicknesses, the dimensional accuracy of the sheet is not significantly adversely affected by the cutting out, so that a manufacturing process simplified by comparison with an extrusion or aluminum casting can be undertaken.

Then, in the bending process for example the third, fourth, fifth and sixth part surface etc. are bent about a bending edge running in one of the parallel trapeze sides at an angle of 90° in relation to the first or the second part surface.

In an especially preferred embodiment the frame elements are made from a sheet steel. In order to fulfil the requirements in relation to stability or vibration behavior, sheet steel with a thickness in the range of 10 mm to 15 mm, preferably 12 mm, is employed.

In embodiments of the present invention, when the frame elements are arranged in their final relative position in relation to one another, the third and the fifth part surface are arranged in parallel opposite one another and the fourth and the sixth part surface are likewise arranged in parallel opposite one another. In other words, in a preferred embodiment of the support frame one part surface of the first frame element and one part surface of the second frame element are always arranged in parallel opposite one another. Opposite one another in this case is to be understood as the part surfaces running in parallel largely overlapping or covering each other.

If the first and the second frame element are put together to form the support frame, the third and the sixth part surface adjoin one another and together form a curved inner surface of the support frame. In the same way the fourth and the fifth part surface adjoin one another and together form a curved outer surface of the support frame. The inner surface of the support frame refers in this case to the part of the lateral area of the support frame, which, in relation to the fictional circle, on the circumference of which the longitudinal axis of the support frame runs, runs closer to the center point of the circle. The outer surface accordingly refers to the part of the lateral area, that is at the greatest distance from the center point of the circle.

As already indicated above, depending on the number of trapezoidal surface segments of a frame element, the part surfaces of the first and second frame element bent over in relation to the first or second part surface are arranged next to or behind one another or nested in one another and in this way, in an exact fit, form the inner surface and outer surface of the support frame. To this extent the dimensions of the trapezoidal surface segments as well as the bent-over part surfaces are matched to one another so that, together in the joined-together position, they form the closed lateral area of the support frame.

In order to further simplify the joining process, in particular a welding of the two frame elements, the trapezoidal surface segments of the first and second part surface each have at least one positioning slot and the third, fourth, fifth and sixth part surface each have at least one positioning lug, wherein a positioning lug engages in a positioning slot in each case. In other embodiments just one or a few positioning lugs or one or a few positioning slots can be provided per frame element. Positioning slots and positioning lugs facilitate the correct positioning of first and second frame element, wherein one positioning lug of the first frame element engages with a positioning slot of the second frame element and vice versa in each case when the first and the second frame element assume their final relative position in relation to one another. As well as this, these ensure a certain hold for the first and second frame element in their desired relative position. In this way positioning slots and positioning lugs speed up and simplify the assembly of the support frame.

In order to achieve a stable connection between first and second frame element, in some embodiments of the curved support frame, the first and second frame element are connected by a welded connection. In this case the welded connection can comprise a continuous, i.e. uninterrupted weld seam between the adjoining part surfaces of the first and second frame element. As an alternative to this the welded connection can comprise individual shorter weld seams, which each cover only a part of the lines of contact between adjoining part surfaces. In yet other embodiments the welded connection can essentially comprise one-dimensional weld points, with which a connection between adjoining part surfaces is established. Mixed forms of various welded connections are conceivable in embodiments. The choice of a suitable form for the welded connection is predominantly directed to the mechanical stability that the support frame must achieve in the joined-together state.

Overall, in accordance with invention, via the reduction of the number of parts to be connected, the outlay in time for the creation of the welded connections is reduced, since overall there has to be less welding. Moreover, in this way a misalignment of the welding of the support frame is reduced.

### BRIEF DESCRIPTION OF THE DRAWINGS

The characteristics, features and advantages of this invention described above, as well as the manner in which these are achieved, will become clearer and easier to understand in conjunction with the description given below of the exemplary embodiments, which are explained in greater detail in conjunction with the drawings. This description does not restrict the present invention to these exemplary embodiments. In various figures the same components are labeled with identical reference characters. As a rule the figures are not true-to-scale. In the figures:

FIG. 1 shows a view of a medical imaging system in the form of an angiography system in accordance with a form of embodiment of the present invention, the support arm of which is embodied with an inventive support frame,

FIG. 2 shows a perspective diagram of an inventive support frame in a version of an embodiment of the present invention in a state joined by a welded connection,

FIG. 3 shows a detailed perspective view of the support frame in accordance with FIG. 2, wherein the second frame element is shown,

FIG. 4 shows another perspective view of the second frame element in accordance with FIG. 3,

FIG. 5 shows a side view of the support frame in accordance with FIG. 2, and

FIG. 6 shows a cross sectional diagram of the support frame in accordance with FIG. 2.

### DETAILED DESCRIPTION

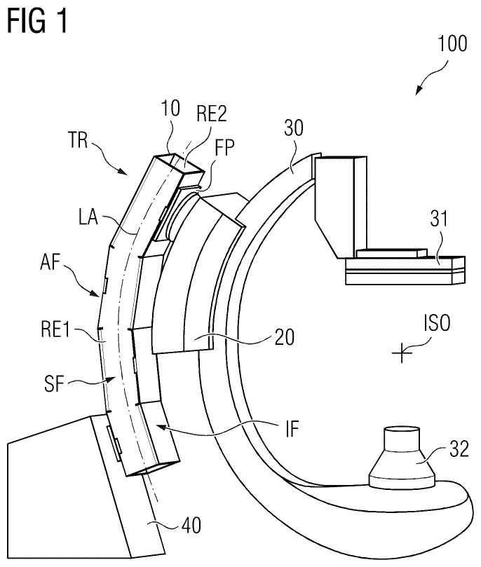

FIG. 1 shows a view of a medical imaging system in the form of an angiography system 100 in accordance with a form of embodiment of the present invention, the support arm of which 10 is embodied with an inventive support frame TR. The angiography system 100 comprises a floor stand 40, via which it is permanently fixed to a foundation and supported. As an alternative the angiography system can also be mounted via a ceiling stand on a ceiling of a room. Arranged rotatably via a gear shaft about the shaft axis on the stand 40 is a support arm 10 of the angiography system 100, so that the support arm 10 is able to be pivoted relative to the stand 40. Arranged on the other end of the support arm in its turn is an intermediate carriage 20 of the angiography system 100 rotatable via a second gear shaft. Located orbitally adjustably in the intermediate carriage is the C-arm 30 of the system, on the end of which an x-ray source 32 and also an x-ray detector 31 are arranged. Via the three degrees of freedom described between stand 40 and C-arm 30 the imaging components x-ray source 32 and x-ray detector 31 can be pivoted very flexibly for acquiring x-ray image data about the isocenter ISO of the system, so that any given regions of the body of a patient can be imaged from any given direction of view.

As already indicated, in the version shown here, the support arm 10 comprises an inventive support frame TR, as will be described in greater detail with reference to the further figures.

In further embodiments of an inventive angiography system 100 not shown in any greater detail however the C-arm 30 or the intermediate carriage 20 can also be embodied comprising an inventive support frame TR.

The support frame TR is a curved support frame, i.e. its longitudinal axis LA runs essentially orbitally, that is along a circular path. In the present example the course of the curved longitudinal axis LA deviates slightly from an ideal orbital path, as will be explained in greater detail below. The support frame TR here has a rectangular cross section and is embodied as a hollow body with a closed lateral area comprising an inner surface IF, an outer surface AF as well as two opposing flat side surfaces SF. Compared with the outer surface AF, the inner surface IF is arranged closer to the isocenter ISO of the angiography system 100. Inner and outer surfaces IF, AF connect the two side surfaces SF to one another. While the side surfaces SF are embodied in one piece and flat in each case, thereby parallel and essentially with the same surface area as one another, the inner and the outer surface IF, AF are embodied by individual part surfaces, which will be discussed in more detail below. Individual part surfaces forming the inner and the outer surface IF, AF and accordingly arranged opposite one another, although likewise parallel and largely coinciding, are not embodied with equal surface areas however.

The support frame TR shown is characterized in accordance with an embodiment of the present invention in that it is constructed from just two parts, namely a first frame element RE1 and a second frame element RE2. The two frame elements RE1, RE2 together form the closed lateral area of the support frame TR.

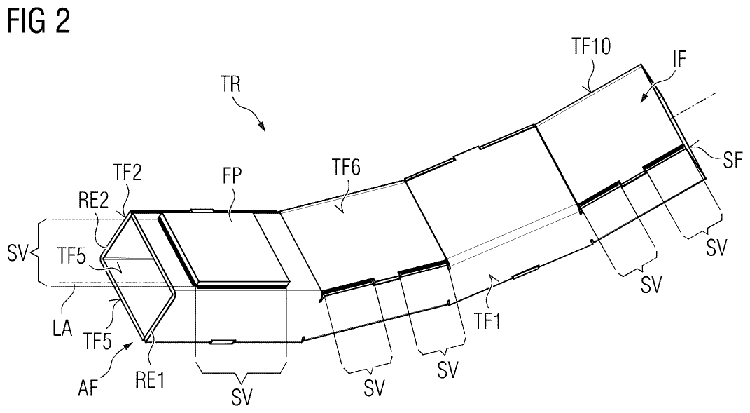

FIG. 2 shows a more detailed perspective diagram of an inventive support frame TR in a version of an embodiment of the present invention in a state joined by welded connection SV.

In other words the support frame TR is manufactured here by the first and the second frame element RE1, RE2 being welded to one another. For this purpose the first and the second frame element RE1, RE2 are made of sheet steel. The welded connection SV in this version is embodied as a plurality of non-contiguous weld seams, which are placed on contact sides between non-adjoining part surfaces of the first and the second frame element RE1, RE2. In this case however the contact sides lying in the inner surface IF and the outer surface AF are not welded. In order to be able to accommodate a gear shaft in a stable manner, a flange plate FP is provided at least in one part surface, said flange plate likewise being welded on all around its circumference from outside.

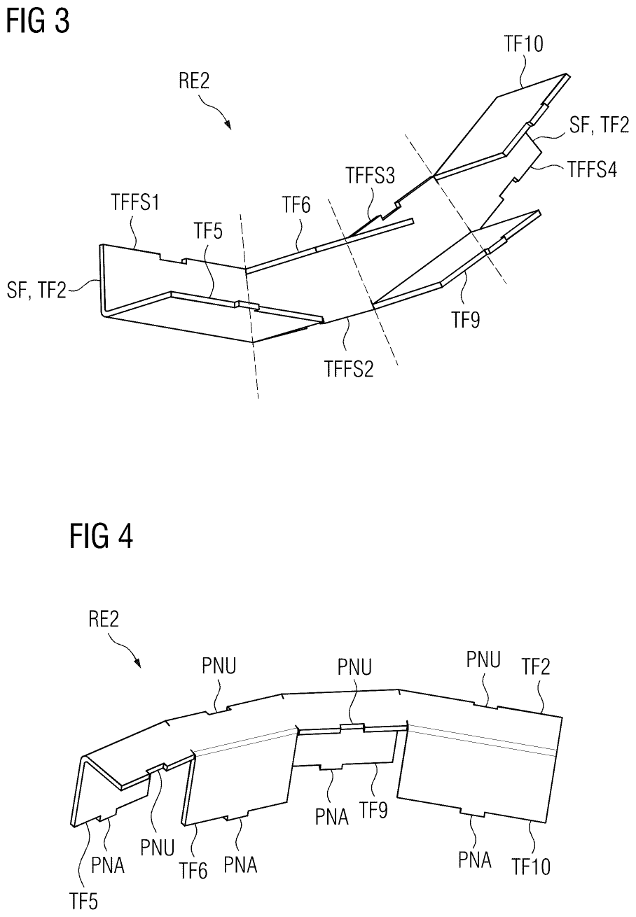

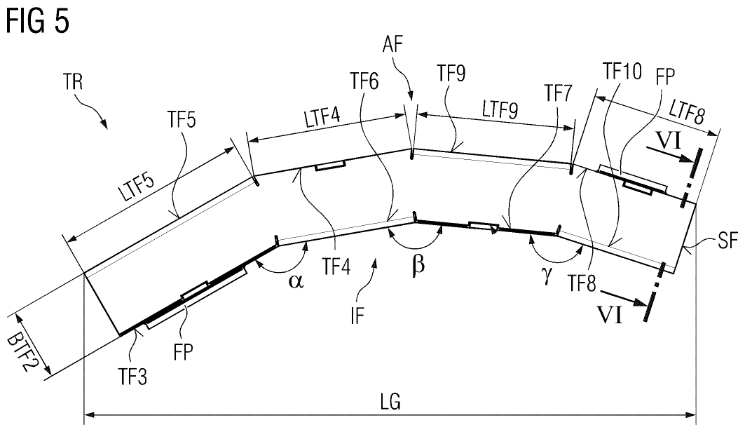

FIG. 3 shows a detailed perspective view of the support frame TR in accordance with FIG. 2, wherein the second frame element RE2 is shown. FIG. 4 shows another perspective view of this second frame element RE2. The second frame element RE2 here is principally also to be understood as being representative for clarifying the structure of the first frame element RE1. Accordingly the first frame element RE1 comprises a first part surface TF1 and the second frame element RE2 comprises a second part surface TF2, wherein the first and the second part surface TF1 and TF2 form opposing parallel side surfaces SF of the support frame TR. First and second part surface TF1, TF2 are each subdivided into at least two trapezoidal surface segments or comprise these. Actually part surface TF2 is subdivided here into four trapezoidal surface segments TFFS1, TFFS2, TFFS3, TFFS4, the adjoining limbs of which are illustrated by the dashed lines.

The curvature of the support frame TR is realized by the limbs of the neighboring trapezes adjoining one another being set at an angle to one another, namely at an angle of between 160° and 167°. Consequently, via the four trapezoidal surface segments TFFS1, TFFS2, TFFS3, TFFS4, the second part surface TF2 forming one side surface SF of the support frame TR is formed in one piece.

In particular the result from FIG. 4 is that the second frame element RE2 comprises at least one fifth and sixth part surface TF5, TF6, which each extend at right angles from second part surface TF2. Actually the second frame element RE2 also comprises further ninth and tenth part surfaces TF9, TF10 likewise at right angles to the second part surface TF2. Similarly the first frame element RE1 comprises at least a third and a fourth, a seventh and an eighth part surface TF3, TF4, TF7, TF8, which each extend at right angles from the first part surface TF1. The part surfaces TF5, TF6, TF9 and TF10 are arranged in this case in such a way that they do not touch one another.

To be exact, the result from the diagram of FIG. 4 is that the fifth, sixth, ninth and tenth part surface TF5, TF6, TF9, TF10 are each connected to one of the trapezoidal surface segments TFFS1, TFFS2, TFFS3, TFFS4 of the second part surface TF2 and have a lateral offset to one another. It can also be easily seen that the second, as well as also the first frame element RE2, RE1 are embodied as bent sheets of metal. In this case the frame element is first laser cut from a planar flat sheet and then bent over at bending edges corresponding to parallel sides of the trapezoidal surface segments TFFS1, TFFS2, TFFS3, TFFS4.

A similar structure is produced in the same way for the first frame element RE1.

The offset of part surfaces TF3 to TF10 extending at right angles from first or second part surface TF1, TF2 makes it possible for the individual part surfaces TF3 to TF10 to be arranged aligned or nested in an exact fit between one another when the frame elements RE1, RE2 assume their final relative position in relation to one another. In this way the result is that the third and the fifth part surface TF3, TF5, the fourth and the sixth part surface TF4, TF6, the seventh and the ninth part surface TF7, TF9 and also the eighth and the tenth part surface TF8, TF10 each lie opposite one another and parallel to one another. This is well illustrated by FIG. 5.

Accordingly the fifth, fourth ninth and eighth part surface TF5, TF4, TF9, TF8 adjoin each other and together form the curved outer surface AF of the support frame TR, while the third, sixth, seventh and tenth part surface TF3, TF6, TF7 and TF8 likewise adjoin each other and together form the curved inner surface IF of the support frame TR.

In order to position the first and the second frame element RE1, RE2, in particular reliably and stably, for the welding process, the trapezoidal surface segments TFFS1, TFFS2, TFFS3, TFFS4 each have at least one positioning slot PNU and the third to tenth part surface TF3 to TF10 each have at least one positioning lug PNA, wherein e positioning lug PNA engages in a positioning slot PNU in each case when the two frame elements RE1, RE2 have assumed their final relative position in relation to one another. The positioning lugs and positioning slots PNA, PNU bring about a fixing of the part surfaces adjoining one another until such time as said surfaces have been welded together along the contact lines.

FIG. 5 shows a side view of the support frame TR in accordance with FIG. 2.

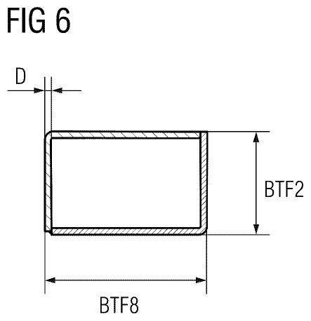

FIG. 6 shows a cross sectional diagram of the support frame TR in accordance with 2.

The focus of the figures here is on the dimensions selected for the individual part surfaces of the frame elements RE1, RE2.

The overall length LG of the support frame TR amounts in the present case to 1572 mm, the width BTF2 of the second part surface TF2, and similarly also of the first part surface TF1 amounts to 188 mm. The lengths LTF5, LTF4, LTF9, LTF8 of the part surfaces TF5, TF4, TF9, TF8 vary slightly and amount to 497 mm, 411 mm, 406 mm and 333 mm. The curvature angles α, B, Y between the trapezoidal surface segments TFFS1, TFFS2, TFFS3, TFFS4 amount to 160°, 165° and 167°. Additionally illustrated in FIG. 6 is the width BTF8 of the eighth part surface TF8, which amounts to 295 mm. This is also identical for the further part surfaces TF3 to TF10. The sheet metal used for the support frame TR has a material thickness D of 12 mm.

Independent of the grammatical term usage, individuals with male, female or other gender identities are included within the term.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of the embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing embodiments. The present invention may, however, be embodied in many alternate forms and should not be construed as limited to only the embodiments set forth herein.

Although the present invention has been illustrated and described in greater detail by the preferred exemplary embodiment, the present invention is not restricted by this exemplary embodiment. Other variations can be derived herefrom by the person skilled in the art without departing from the scope of protection of the present invention.

What is claimed is:

1. A curved support frame for an angiography system, the curved support frame comprising:
a hollow body having a first frame element and a second frame element, wherein the first frame element and the second frame element together form a closed lateral area of the curved support frame.

2. The curved support frame as claimed in claim 1, wherein a curvature of the curved support frame extends along a longitudinal axis of the curved support frame.

3. The curved support frame as claimed in claim 1, wherein
the first frame element includes a first part surface,
the second frame element includes a second part surface, and
the first part surface and the second part surface are opposite parallel side surfaces of the curved support frame.

4. The curved support frame as claimed in claim 3, wherein the first part surface and the second part surface each include at least two trapezoidal surface segments.

5. The curved support frame as claimed in claim 4, wherein neighboring trapezoidal surface segments of at least one of the first part surface or the second part surface are arranged at an angle ranging from 155° to 175° relative to each other.

6. The curved support frame as claimed in claim 4, wherein
the first frame element includes at least a third part surface and a fourth part surface, the third part surface and the fourth part surface extending at right angles from the first part surface, and
the second frame element includes at least a fifth part surface and a sixth part surface, the fifth part surface and the sixth part surface extending at right angles from the second part surface.

7. The curved support frame as claimed in claim 6, wherein
the third part surface and the fourth part surface are each connected to a trapezoidal surface segment of the first part surface, and
the fifth part surface and the sixth part surface are each connected to a trapezoidal surface segment of the second part surface.

8. The curved support frame as claimed in claim 6, wherein
the third part surface and the fifth part surface are arranged opposite one another in parallel, and
the fourth part surface and the sixth part surface are arranged opposite one another in parallel.

9. The curved support frame as claimed in claim 6, wherein
the third part surface and the sixth part surface adjoin one another and together form a curved inner surface of the curved support frame, and
the fourth part surface and the fifth part surface adjoin one another and together form a curved outer surface of the curved support frame.

10. The curved support frame as claimed in claim 6, wherein
the trapezoidal surface segments each include at least one positioning slot, and
the third part surface, the fourth part surface, the fifth part surface and the sixth part surface each include at least one positioning lug, wherein each of the at least one positioning lug engages in a positioning slot.

11. The curved support frame as claimed in claim 1, wherein the first frame element and the second frame element are connected by a welded connection.

12. The curved support frame as claimed in claim 1, wherein the first frame element and the second frame element are bent sheets.

13. An angiography system comprising a curved support frame as claimed in claim 1.

14. The angiography system as claimed in claim 13, further comprising:
a C-arm including the curved support frame.

15. The angiography system as claimed in claim 13, further comprising:
a support arm including the curved support frame.

16. The curved support frame as claimed in claim 2, wherein
the first frame element includes a first part surface,
the second frame element includes a second part surface, and
the first part surface and the second part surface are opposite parallel side surfaces of the curved support frame.

17. The curved support frame as claimed in claim 5, wherein
the first frame element includes at least a third part surface and a fourth part surface, the third part surface and the fourth part surface extending at right angles from the first part surface, and
the second frame element includes at least a fifth part surface and a sixth part surface, the fifth part surface and the sixth part surface extending at right angles from the second part surface.

18. The curved support frame as claimed in claim 7, wherein
the third part surface and the fifth part surface are arranged opposite one another in parallel, and
the fourth part surface and the sixth part surface are arranged opposite one another in parallel.

19. The curved support frame as claimed in claim 7, wherein
the third part surface and the sixth part surface adjoin one another and together form a curved inner surface of the curved support frame, and
the fourth part surface and the fifth part surface adjoin one another and together form a curved outer surface of the curved support frame.

20. The curved support frame as claimed in claim 8, wherein
the third part surface and the sixth part surface adjoin one another and together form a curved inner surface of the curved support frame, and
the fourth part surface and the fifth part surface adjoin one another and together form a curved outer surface of the curved support frame.
